(19) United States
(12) Patent Application Publication
(10) Pub. No.: US 2006/0173514 A1
(43) Pub. Date: Aug. 3, 2006

(54) Wound treatment device for photodynamic therapy and method of using same

(75) Inventors: Merrill A. Biel, Minneapolis, MN (US); Chester E. Sievert JR., Minneapolis, MN (US)

Correspondence Address: JOHN F. KLOS;Fulbright & Jaworski LLP, Suite 2100, 80 South 8th Street, Minneapolis, MN, 55402, US

(73) Assignee: Advanced Photodynamic Technologies, Inc.

(21) Appl. No.: 11/050,349

(22) Filed: Feb. 2, 2005

(51) Int. Cl. A61N 5/06

(52) U.S. Cl. 607/88; 607/89

(57) ABSTRACT

The invention relates to a light emitting treatment device including one or more light members, which are configured to emit light energy for the purpose of performing localized photodynamic therapy at a targeted field. The light members may be disposed in a substantially uniform array and be configured to emit energy in a substantially uniform pattern. The light treatment device has a self-contained energy supply. The light emitting treatment device may be controlled to deliver one or more various light doses and dose rates at various light frequencies per treatment. The treatment device may be made of a polymeric material configured to conform to a body surface. The treatment device may contain the photosensitizer. The light emitting treatment device may further include a heat dissipating layer such as a layer of gold or gold alloy, or a layer of adhesive disposed on at least one of the one or more surfaces. Methods of using the treatment device are also disclosed.

### BACKGROUND OF THE INVENTION

The invention relates to a medical device for photodynamic therapy (PDT). More specifically, the invention relates to a flexible multi-element dressing composed of; polymeric, reflective and diffusion layers, a light delivery source and an energy source. The present invention advantageously uses light energy to treat or detect pathologies of living tissue, especially at wound sites. The present invention may contain or be used in combination with photosensitizing agents and surface-acting agents.

The worldwide rise in drug resistant bacteria and fungi that infect wounds and burns has led to the search for alternative methods of selectively destroying microorganisms without harming the host tissue. Because an infection is initially contained to the wound, one method of selectively killing microorganisms may be the combination of photosensitive materials and visible light, known as photodynamic therapy (PDT). PDT uses photosensitive materials that preferentially accumulate in microorganisms, virulence factors and cancer cells. Subsequent illumination with light of the appropriate wavelength excites molecules of the photosensitive material to the excited singlet or triplet states that oxidize many biological molecules include proteins, nucleic acids and lipids, leading to cytotoxicty. Hence, PDT selectively destroys microorganisms, virulence factors or cancer cells without destroying the host tissue. PDT may also be used prophylactically to prevent an infection.

The field of topical PDT and medical devices for practicing photodynamic therapy are known. In one approach, various types of pads, patches, or garments containing light-emitting elements (or having light-emitting elements attached thereto) are placed in contact with the skin or other tissue of the patient to irradiate that portion of the skin or tissue with light. The light may itself provide a therapeutic benefit due to its characteristic wavelengths, or may act in combination with a pharmacological agent (which is applied topically to the patient's skin or tissue, or is injected or ingested by the patient), which reacts with the light and produces a therapeutic benefit. The pharmacological agent may accumulate in the region being treated, or may react upon exposure to the light at the exposed region while traversing within the circulatory system. Representative examples of pads, patches, garments, or shaped objects that contain or carry light-emitting elements for use in photodynamic therapy are known.

The process of iontophoresis has found use in the delivery of ionically charged therapeutic agent molecules such as pilocarpine, lidocaine and dexamethasone. In this delivery method, ions bearing a positive charge are driven across the skin at the site of an electrolytic electrical system anode, while ions bearing a negative charge are driven across the skin at the site of an electrolytic system cathode. Some iontophoretic devices have been constructed of two electrodes attached to a patient, each connected by a wire to a remote power supply, generally a microprocessor-controlled electrical instrument. Because they involve direct patient contact with the electrodes, these devices are most conveniently constructed so as to make use of disposable electrodes, associated with a reusable electric instrument. The electrical instruments generally are battery powered and designed in a manner such that the batteries can be easily replaced as they become consumed.

More recently, self-contained wearable iontophoretic systems have been developed. These systems are advantageous in that they do not have external wires and are much smaller in size. Examples of such systems can be found in a variety of U.S. patents, including U.S. Pat. Nos. 4,927,408; 5,358,483; 5,458,569; 5,466,217; 5,533,971; 5,605,536; 5,651,768; and 5,685,837. Depending on factors relating to cost, particular use and convenience, wearable iontophoretic systems can be “reusable” or “disposable”. Reusable systems may be defined as systems in which the power source is designed to be replaceable; whereas disposable systems may be defined as devices in which the entire iontophoretic system is designed to be disposed following a single use or consumption of the original power source.

The power sources for self-contained iontophoretic systems can further be characterized as “galvanic”, “electrolytic” or a combination of these. “Galvanic” power is defined as power supplied by a couple, including a pair of electrodes having amounts of dissimilar surface electroactive materials that inherently provide a voltage difference between the electrodes (anode and cathode) and which normally are connected directly by a conductor. “Electrolytic” power sources are power sources generally remote from but in conductive contact with the electrodes, and usually include such devices as button-type batteries or sheet-like multi-layer elements. Electrolytic and galvanic sources of power are known in the art and describe, for example, in the above-referenced U.S. Pat. Nos. 4,927,408; 5,533,971; and 5,685,837.

With iontophoresis, the rate that medications are introduced is a function of the level of current, while the total quantity of medication delivered is a function of both current level(s) and time or the amount of total charge transferred. Because of this relation, often the quantity of medication introduced by iontophoresis is referred to in units of mA-minutes of dosage. Thus, for example, an equivalent 40 mA-minute dosage can be delivered at different rates; 0.1 mA for 400 minutes, 1 mA for 40 minutes, 10 mA for 4 minutes, etc.

Control of the dosage delivered by iontophoresis is usually accomplished by means of electrical circuitry in the form of electrical components mounted on the circuit layer. Electrical components can be utilized to regulate the level, waveform, timing and other aspects of the electrical current and the system usually includes a microprocessor adapted to control the current over time. These electrical circuits are well known and are described, for example, in U.S. Pat. No. 5,533,971. Electronic means have been developed to regulate the total iontophoretic dosage in its delivery-time profile by precise, pre-determined control of the charge capacity of the power supply design.

### SUMMARY OF THE INVENTION

The invention is a self-contained photodynamic therapy (PDT) wound treatment device for delivering light from one or more light-emitting elements through a flexible dressing that conforms to the skin or tissue of the patient. A polymer or copolymer based dressing such as a hydrogel and/or hydrocolloid is particularly well suited as the patient contact medium of the present invention.

In one embodiment, the light-emitting treatment device is a self-contained device including a light source, flexible circuitry, diffusion layers, reflective layers, energy source and fabric cover connected to the flexible dressing. The device may be adhered to a wound site by an adhesive provided upon the dressing's perimeter.

In another embodiment of the present invention, an iontophoretic drug delivery system may be incorporated into the self-contained device. A variety of different pharmaceutical compounds may be introduced via iontophoresis, including but not limited to, anti-inflammatory drugs, analgesics, anesthetics, surfactants, and certain photosensitive materials.

The invention further includes a method of using a light-emitting treatment device. In one embodiment, the method includes identifying an area of treatment on a body surface and providing a surface acting agent and/or photosensitive material to the wound site. In another embodiment, the method includes incorporating the surface acting agent and/or photosensitive material into the flexible dressing to allow for a release of the compounds to the wound site.

Still other representative embodiments and advantages of the present invention and methods of construction of the same will become readily apparent to those skilled in the art from the following detailed description, wherein only the preferred embodiments are shown and described, simply by way of illustration of the best mode contemplated for carrying out the invention. As will be realized, the invention is capable of other and different embodiments and methods of construction, and its several details are capable of modification or adaptation in various respects all without departing from the invention as disclosed and claimed. Accordingly, the appended drawings and description contained herein, as well as the descriptions and drawings contained in the applications and associated documents to which the benefit of priority has been claimed and which are incorporated herein by reference as though fully set forth, are to be regarded as illustrative in nature and not as restrictive or limiting.

### BRIEF DESCRIPTIONS OF THE DRAWINGS

Preferred embodiments of the invention will be described in detail hereinafter with reference to the accompanying drawings, in which like reference numeral refer to like elements throughout, wherein:

FIG. 1 is a depiction of a patient with an embodiment of the light emitting treatment device of the present invention.

FIG. 2 is a partially broken away perspective view of an embodiment of the present invention.

FIG. 3 is a cross-section of the device of FIG. 2.

FIG. 4 is a bottom plan view of the device of FIG. 2.

### DETAILED DESCRIPTION OF THE INVENTION

The present invention may be used in conjunction with or in relation to inventions disclosed in the following applications of the applicant, including:
Dye Treatment Solution and Photodynamic Therapy and Method of Using Same, U.S. Pat. No. 6,251,127;
Method of Enhancing Photodynamic Therapy by Administering an Immunologic Adjuvant, Ser. No. 09/139,861;
Methylene Blue and Toluidine Blue Mediated Fluorescence Diagnosis, Pat. No. 6,083,487;
Photodynamic Therapy Utilizing a Solution of Photosensitizing Compound and Surfactant, Ser. No. 09/514,070;
Photodynamic Cellular and Acellular Organism Eradication Utilizing a Photosensitive Material and Surfactant, Ser. No. 09/792,578;
Photodynamic Cellular and Acellular Organism Eradication Utilizing a Photosensitive Material and Benzalkonium Chloride, Ser. No. 10/026,198; and
Apparatus and Method of Photodynamic Eradication of Organisms Utilizing Pyrrolnitrin, Ser. No. 10/052,990, now U.S. Pat. No. 6,623,513.

All information within these patents and applications is incorporated by reference herein for all purposes.

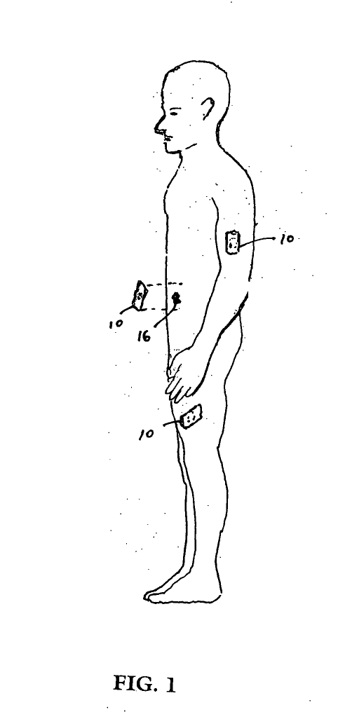

Referring to FIG. 1, an embodiment of the present invention is generally indicated by numeral 10 and is illustrated as applied at a wound site on a human arm and leg. The device 10 may find application to other internal or external sites of a human or other animal. Various preferred embodiments of the light-emitting photodynamic treatment device of the present invention are described below, with the light-emitting photodynamic treatment device being generally referenced herein by the numeral 10.

The light-emitting photodynamic treatment device 10 is particularly adapted to be placed in conforming contact with the patient's body and irradiate a region of the skin, tissue, or other external, exposed, or internal organs of the patient's body, and used to provide topical or surface photodynamic therapy (PDT) to that region or surface, including PDT which requires applying light energy for long periods. Hereafter, the terms “skin” and “tissue” will be used interchangeably or alternately, and the external skin, external organs, exposed internal tissue surfaces, and internal tissue or organs may be referred to collectively and interchangeably as “skin” or “tissue.” The term “tissue” is further understood to broadly encompass the skin or any other body surfaces to which the light-emitting photodynamic treatment device 10 would be applied on or within a patient, including exposed or externally-accessible regions of the patient's body, or regions of the patient's body requiring an invasive procedure.

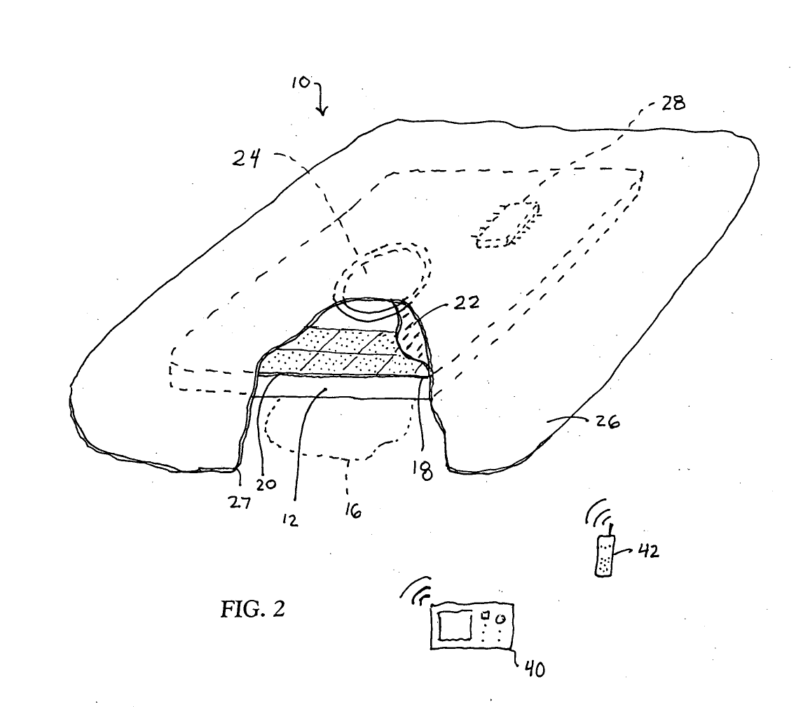

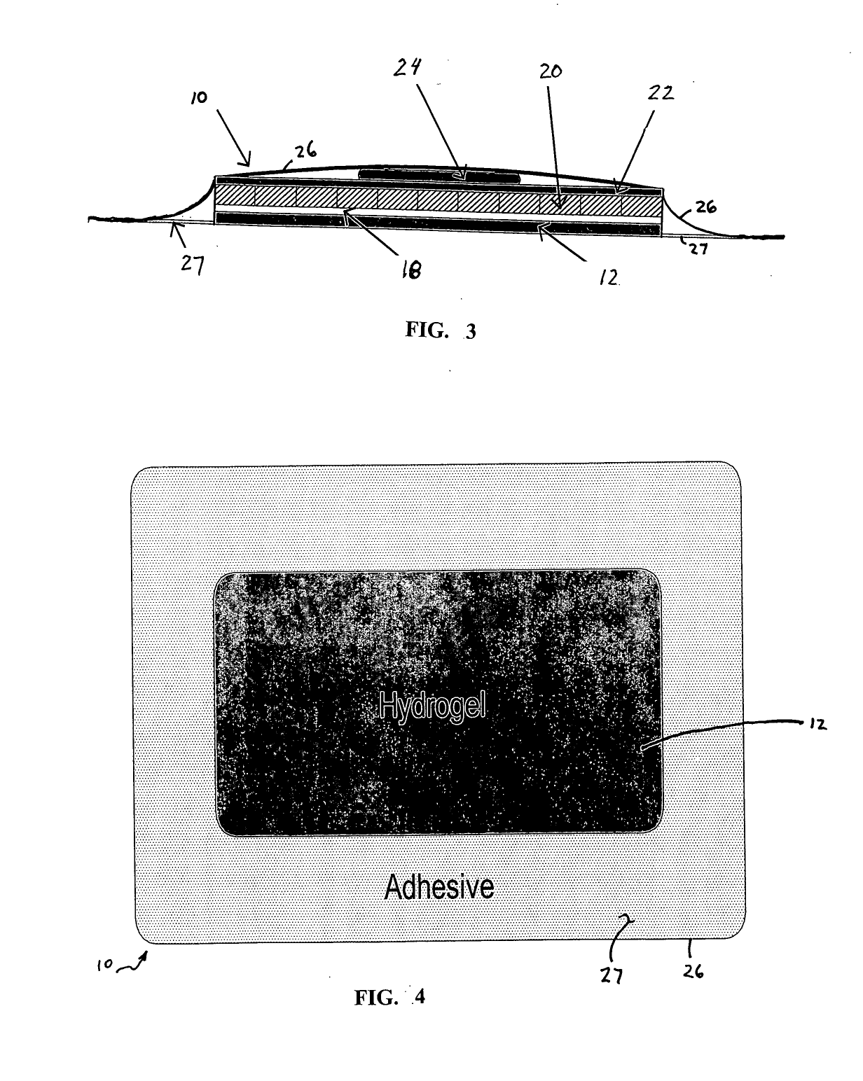

FIG. 2 is a partially broken-away perspective view of one embodiment of the present invention including a flexible dressing 12 in contact with the tissue surface proximate to the wound site 16. Assembly 10 further includes a light diffusive layer 18, a light source 20, a light reflector 22, microprocessor controlled flexible circuitry, a battery 24 and a flexible fabric cover 26. Assembly 10 may optionally further include a heat dissipative element operatively coupled to light source 20 to transfer heat away from the tissue surface. Heat dissipative layer may be a conductive layer or similar element contained within assembly 10 and transferring heat generated by light source 20 away from the tissue surface. An adhesive 27 is provided upon portions of the fabric cover 26 and may be used to adhere the device 10 at the wound site 16. An optional electronic controller 28 is also illustrated. FIG. 3 is a bottom plan view of the device 10 illustrating a polymer or copolymer based dressing such as a hydrogel 12 and an adhesive 27. FIG. 4 illustrates a cross-sectional view of the light-emitting treatment device 10.

Flexible dressing 12 may or may not have a polymer or copolymer such as a hydrogel or a hydrocolloid or foam or a combination thereof as the dressings in contact with the wound site. Hydrocolloids and hydrogels are well know and the selection of a particular dressing 12 for application in the present invention would be within the capacity of one of ordinary skill in the relevant arts.

Hydrocolloids are a type of dressing containing gel-forming agents, such as sodium carboxymethylcellulose (NaCMC) and gelatin. In the presence of wound exudate, hydrocolloids absorb liquid and form a gel, the properties of which are determined by the nature of the formulation. Some dressings form a cohesive gel, which is largely contained within the adhesive matrix; others form more mobile, less viscous gels, which are not retained within the dressing structure. In the intact state, most hydrocolloids are impermeable to water vapor, but as the gelling process takes place, the dressing becomes progressively more permeable. The loss of water through the dressing in this way enhances the ability of the product to cope with exudate production. One feature of hydrocolloids that is appreciated by clinicians is wet tack; unlike most dressings, they can adhere to a moist site as well as a dry one.

Hydrocolloid dressings contain a gel-forming agent, which is activated when a wound exudate comes in contact with it. The gel becomes progressively more permeable to water, allowing water vapor to pass through. In this way, small amounts of drainage can be effectively handled by a wound dressing which needs to be changed less frequently.

A gel is a three-dimensional polymeric network that has absorbed a liquid to form a stable, usually soft and pliable, composition having a non-zero shear modulus. The liquid contributes a substantial percent of the overall volume of the composition. When the liquid is water, the gel is called a hydrogel. Due to their unique composition, i.e., largely water absorbed into a biologically inert polymeric matrix, hydrogels have found use in numerous biomedical applications. They are also used as wound dressings, both with and without incorporated medicaments that can be released from the matrix to aid in the healing process (U.S. Pat. Nos. 3,963,685 and 4,272,518, incorporated by reference herein). In addition, hydrogels have found substantial use as vehicles for the sustained release of biologically active substances.

The use of hydrogels in the treatment and management of burns and wounds is well known in the art. Hydrogel dressings are desirable, in part, because they provide protection against infectious agents. Hydrogel dressings are further desirable because wound exudate does not generally dry and consolidate with hydrogels or hydrogel laminates. Consequently, removal of a hydrogel dressing is usually neither painful nor detrimental to the healing process. U.S. Pat. No. 4,438,258, incorporated by reference herein, relates to hydrogels that may be used as interfaces between damaged skin tissue and its external environment. As disclosed therein, hydrogels may be polymerized about some type of support, such as a mesh of nylon, used as an unsupported film, spun in fibers and woven into a fabric, or used as a powder. Further, hydrogels may be used to provide a controlled release of a medical composition. U.S. Pat. No. 4,552,138 discloses a wound dressing material of at least one layer of a polymeric, hydrophilic gel wherein the gel is cross-linked and acetalized with formaldehyde. U.S. Pat. No. 4,567,006 discloses a moisture vapor permeable, adhesive surgical dressing comprising a continuous film of a hydrophilic polymer. Such a dressing is suitable for use on moist wounds because it allows water to evaporate rapidly from the wound area in the presence of an excess of exudate but, as the amount of exudate diminishes, so does the rate of evaporation. The resulting amount of exudate is enough to keep the wound moist without causing blistering of the dressing.

Preferably, the polymer or coploymer 12 is generally transparent or translucent to wavelengths of the light source 20. In the illustrated embodiment, a separate diffusive layer 18 is provided. In alternative embodiments, the diffusive layer 18 may be eliminated and light diffusion may be provided by the polymer or coploymer 12, such as by incorporation of titanium dioxide within the polymer or coploymer 12. In the illustrated embodiment, the diffusive layer 18 is a thin film.

Together, the dressing 12 and fabric cover 26 define the general shape of the light-emitting treatment device 10 and form an integral or unitary structure which will not separate from one another when flexed or stretched sufficiently for application to the intended region of the patient's body.

Light reflector 22 is optional and may include a light reflective layer. Light reflector 22 is used to reflect light emitting from the light source 20 back toward the wound site. In an embodiment of the present invention, the light source 20 may be oriented toward the reflector 22 so that light passes through an increased effective thickness of translucent polymer or coploymer 12. In this manner, the diffusion of light from light source 20 may be increased. In other embodiments, light reflector 22 may be incorporated into the light source 20 and provided as a layer or elements within light source 20.

In the illustrated embodiment of the light-emitting photodynamic treatment device 10, light source 20 includes a plurality of light-emitting elements include vertical cavity surface-emitting lasers (VCSEL's) arrayed in a pattern or configuration on a flexible circuit board as desired and operatively coupled to battery 24 using any suitable conductors.

Together, the dressing 12 and fabric cover 26 define the general shape of the light-emitting treatment device 10 and form an integral or unitary structure which will not separate from one another when flexed or stretched sufficiently for application to the intended region of the patient's body.

In the illustrated embodiment, light source 20 includes a plurality of VSCEL elements. In alternative embodiments, light source 20 may include one or more LED's, organic light emitting diodes (OLED's), laser diodes, light emitting plastics, and chemoluminescent materials. The wavelengths of light emitted by the light source may be variable and may be controlled by an internal or external controller. The light source 20 may be pulsed on and off during a treatment, with the frequency of the on/off cycles ranging from nanoseconds to hours.

In the illustrated embodiment, battery 24 is a single battery element. In alternative embodiments, battery 24 may include a plurality of battery elements. Battery 24 may be rechargeable via direction connection to an external power supply, radio frequency or via electromagnetic induction. Battery 24 may be controlled to maximize efficiency. The discharge of battery 24 may be controlled by an internal controller 28 so that the light intensity of light source 20 is substantially uniform during a treatment. In another embodiment, controller 28 may vary the light intensity of light source 20 during the treatment period. The waveform of the light intensity may include ramps, pulses, or other shapes. Those of ordinary skill in the art will appreciate that many types of batteries may be utilized, including but not limited to galvanic, chemical, capacitive battery technologies. Battery 24 may include one-time use or rechargeable devices. Battery 24 is to be broadly defined to include alternative energy sources such as capacitors, piezoelectric systems, chemoluminescent devices, solar powered devices, etc.

Controller 28 is optional and may perform a variety of device 10 functions. Controller 28 may be programmed to control the wavelengths, waveform and/or pulse durations of light source 20. Controller 28 may include a communications component for communicating information to a remote transceiver 40, such as a laptop computer. The communications component may include an antenna and transceiver and utilize known communications protocols, for example Blue Tooth. Controller 28 may include a memory element to store information relating to the device 10 use, such as time stamp information, dose rates, light doses, etc. Controller 28 may control the release of photosensitive material from a reservoir within device 10. Controller 28 may be controlled by a remote controller 42 via wireless communication. Controller 28 may be activated by a user-accessible ON/OFF button. Controller 28 may also receive signals from a photodetector element, such as a photodiode, to control the light source. For example, the photodetector signals may be utilized by controller 28 to terminate the application of light from light source 28 upon reaching a predetermined light dose at the tissue site. The photodetector element is optional and may be incorporated within or above the dressing relative to the tissue surface depending upon the particular configuration of the light source 20.

The fabric layer 26 preferably provides a moisture and microbe barrier. A variety of different fabrics (woven or non-woven) could be utilized in device 10. An adhesive 27 preferably secures the fabric layer 26 to a patient's skin or tissue surface. A variety of biomedical adhesive would be practicable to adhere the device 10 to the patient.

Photosensitizers useful in the described methods can be prepared or formulated for administration in any medium known to the skilled artisan including, but not limited to, tablet, solution, gel, aerosol, dry powder, biomolecular matrix. Photosensitizers useful in the new methods can be administered to a subject by any means known to the skilled artisan including, but not limited to, oral, systemic injection (e.g., intramuscular, intraperitoneal, subcuticular, venous, arterial, lymphatic etc.), topical delivery, topical delivery by a medium (e.g., slow release formulations via photosensitizer impregnated hydrogel polymers), inhalation delivery (e.g., dry powder, particulates), microspheres or nanospheres, liposomes, erythrocyte shells, implantable delivery devices, local drug delivery catheter, perivascular delivery, pericardial delivery, eluting stent delivery. Photosensitizers can also be conjugated to targeting agents, such as antibodies directed to specific target tissues (e.g., tumor-associated antigens or vascular antigens, such as the ED-B domain) and microorganisms (e.g., bacteria, viruses, fungi, and microbial virulence factors). Ligands directed against receptors that are up-regulated in tumor cells can also be conjugated to photosensitizers. For example, low-density lipoprotein (LDL) can be conjugated to photosensitizers to be directed at tumor cells that express the LDL receptor, and estrogen can be used to target photosensitizers to estrogen receptor expressing cells, such as found in hormone-dependent tumors. Liposomes and immunoliposomes can also be used as targeting agents to carry the photosensitizers to specific target tissues and microorganisms.

A photosensitive material is defined herein as a material, element, chemical, solution, compound, matter, or substance which is sensitive, reactive, receptive, or responsive to light energy. Photosensitive materials may be provided in a liquid, gaseous, or solid form, including but not limited to liquids, solutions, topical ointments, or powders. Photosensitive materials for use in accordance with the present invention are generally non-toxic to the target cellular or acellular organisms and surrounding tissues at concentrations envisaged. However, there is no particular requirement that the photosensitive material should be non-toxic to the microbes. Particular photosensitizers, which may be used in accordance with the invention, include dyes and compounds such as methylene blue and toluidene blue.

The terms “chemical agent” and “surface-acting agents” and “surfactants” as used herein are broadly defined to include materials, compounds, agents, chemicals, solutions, or substances, which alter the energy relationships at molecular interfaces. Among the manifestations of these altered energy relationships is the lowering of surface or interfacial tensions. Chemical agents or compounds displaying surface activity are characterized by an appropriate structural balance between one or more water-attracting groups and one or more water-repellent groups. Surfactants are characterized by having two different moieties, one polar and the other nonpolar. The polar moiety is referred to as hydrophilic or lipophobic, and the nonpolar as hydrophobic or lipophilic. The electrical charge on the hydrophilic portion of a surface acting agent may serve as a convenient basis of classification of these compounds. Surface-active agents have been classified as: Anionic, Cationic, Non-Ionic, and Amphoteric. Other classes of surfactants are also known or may be developed or defined in the future. Chemical agents, such as surfactants, are known to affect the permeability of cell membranes, and membrane-like structures of acellular organisms, such capsids and envelopes. The ability of these chemical agents or surfactants to become oriented between lipid and protein films is thought to produce a disorientation of the membrane of microorganisms, so that it no longer functions as an effective osmotic barrier. The term ‘membrane’ as used herein is meant to broadly include cellular or acellular organism structures, such as cell walls, cytoplasmic membranes, cell envelopes, coverings, capsids, envelopes, or other types of boundary-defining terms of cellular or acellular organisms. It is believed that a photosensitive material may diffuse through the membrane of a microorganism having a surfactant-compromised membrane. A photosensitive material concentration within the membrane and the organism increases over time via osmotic diffusion of the photosensitive material across the surfactant-compromised membrane. The polymixins, colisimethate, and the polyene antifungal agents nystatin and amphotericin are surfactants, as is sodium dodecyl sulfate (SDS). Cetrimide is also a known surfactant.

A surface-acting agent may be provided at or near the tissue surface before or after application of the device 10 to the tissue surface. The surface-acting agent may be benzalkonium chloride provided in a concentration range of between 0.001% to 1%. More particularly, the surface acting agent may contain benzalkonium chloride in a concentration range of between 0.005% to 0.05%. The surface acting agent also contains polymyxin B sulfate or cetrimide or a combination of both.

In one embodiment of the invention, the photosensitive material and/or surface-acting agents are incorporated into the dressing 12. Dressing 12 may then slowly release these photosensitive material and/or surface-acting agents during a treatment. Absorption, impregnation or other technologies used to incorporate these compounds into the dressing 12 would be apparent to those of ordinary skill in the relevant arts.

In another embodiment of the invention, an iontophoretic drug delivery system may be incorporated into the device 10. Iontophoresis is a percutaneous absorption-promoting system which employs electricity for external stimulation. Its principle is such that skin barrier permeability of drug molecules is promoted by movements of positively-charged molecules from an anode to a cathode and those of negatively-charged molecules from the cathode to the anode in an electric field mainly produced between the anode and the cathode by power supply. Thus, in iontophoresis, an anode and a cathode are provided in pair and a current is generated between the anode and cathode, thereby moving a drug. A constant current control unit may be employed so that a current can be maintained at a predetermined value irrespective of an impedance difference due to individual difference.

Electrodes for the iontophoretic drug delivery system may be positioned within or upon dressing 12. The drug may be incorporated within dressing 12, or may be separately contained and released during application of device 10. Preferably the iontophoretic drug delivery system is used to introduce the surfactant(s) and/or photosensitive material(s) deeper into a tissue site. Current discharge through the electrodes may be controlled by a microprocessor or microcontroller. The power supply for the iontophoretic drug delivery system may include one or more cells. Additional details of an iontophoretic drug delivery system are disclosed in U.S. Pat. No. 6,653,014, incorporated by reference herein for all purposes.

The rate that surfactants and/or photosensitive materials are introduced is a function of the level of current, while the total quantity of medication delivered is a function of both current level(s) and time or the amount of total charge transferred. Because of this relation, often the quantity of medication introduced by iontophoresis is referred to in units of mA-minutes of dosage. Thus, for example, an equivalent 40 mA-minute dosage can be delivered at different rates; 0.1 mA for 400 minutes, 1 mA for 40 minutes, 10 mA for 4 minutes, etc. It is envisioned that a current density of between 0.15-0.60 m-A/cm² may find applicability within a system according to the present invention.

Control of the dosage delivered by iontophoresis is usually accomplished by means of electrical circuitry in the form of electrical components mounted on the circuit layer. Electrical components can be utilized to regulate the level, waveform, timing and other aspects of the electrical current and the system usually includes a microprocessor adapted to control the current over time. These electrical circuits are well known and are described, for example, in U.S. Pat. No. 5,533,971. Electronic means have also been developed to regulate the total iontophoretic dosage in its delivery-time profile by precise, pre-determined control of the charge capacity of the power supply design.

Operation of the embodiment of the Invention:

A method of utilizing the device 10 includes the steps of administering a photosensitive material to a tissue site; adhering the device 10 at the tissue site so that dressing 12 overlays the wound; and illuminating the tissue site with the light source 20 to provide a therapeutic photodynamic reaction of the photosensitive material at the wound.

The light source 20 may provide a light dosage rate of between 1 mW/cm² and 200 mW/cm². In another embodiment, light source 20 may provide a light dosage rate of between 1 mW/cm² and 20 mW/cm². In yet another embodiment, the light source 20 may provide a light dosage rate of between 5 mW/cm² and 20 mW/cm². The light emitting treatment device 10 may provide light wavelengths ranging from about 380 nm to about 900 nm.

The light source 20 may be controlled to provide a low frequency pulsed light to the wound site. Light source 20 may be activated and/or deactivated in a number of different ways. For example, a user-accessible switch or a remotely controlled switch can be utilized to activate light switch 20. The pulsed light may include an alternating high intensity light and a substantially reduced intensity light. The light source may include an ON state and an OFF state, with the ON state providing a light dosage rate of between 1 mW/cm² and 200 mW/cm² and the OFF state providing a light dosage rate of less than 10 mW/cm². Preferably, the light dosage rate of the ON state is substantially greater than the light dosage rate during the OFF state. During the ON state of operation, the light source may be characterized by a first duty cycle. As used herein, the term “duty cycle” means the ratio of the on time of the light source to the sum of the on and off times. Furthermore, the ON and OFF states may be characterized by a second duty cycle defined by the ratio of the time in the ON state to the sum of the time in both ON and OFF states. In this regard, the light source may be continuously pulsed on and off with varying time intervals between on/off transitions so that during both the ON state and the OFF state, the light source is both on and off. Additional details of a low frequency pulsed light source are disclosed in the applicant's co-pending U.S. patent application entitled “Photodynamic Therapy Utilizing Low Frequency Light Modulation”, Ser. No. ______ and filed on Feb. ______, 2005, and incorporated by reference herein for all purposes and teachings.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. An assembly comprising:
a flexible dressing adapted to contact a tissue surface;
a light source being powered by an energy source, said light passing through at least a portion of the dressing to illuminate the tissue surface, and said light promoting a therapeutic photodynamic reaction of a photosensitive material; and
an adhesive element for adhesively securing the dressing to the tissue surface.

2. The assembly of claim 1 wherein the flexible dressing is a polymer or copolymer or a silicone or a foam or a combination thereof.

3. The assembly of claim 1 wherein the photosensitive material is either incorporated within the flexible dressing or provided separately from the dressing.

4. The assembly of claim 1 further comprising a light diffuser, said light diffuser being incorporated within the dressing or being an element separate from the dressing.

5. The assembly of claim 4 wherein the flexible dressing has non-uniform diffusivity so that the light intensity applied to the tissue surface is non-uniform.

6. The assembly of claim 1 wherein the adhesive element extends beyond at least part of the flexible dressing.

7. The assembly of claim 1 further comprising: a reflective element for reflecting light from the light source back toward the tissue surface, said light source being disposed between the reflective layer and the tissue surface.

8. The assembly of claim 7 wherein the reflective element is a reflective layer or a reflective portion of the light source.

9. The assembly of claim 1 wherein the light source is a sheet illuminator.

10. The assembly of claim 1 wherein the light source includes a plurality of VCSEL elements.

11. The assembly of claim 1 wherein the light source is an LED or an OLED or a laser diode or a light emitting plastic or a chemoluminescent material or a combination thereof.

12. The assembly of claim 1 wherein the light source provides a light dosage rate of between 0.1 mW/cm² and 200 mW/cm².

13. The assembly of claim 11 wherein the light source provides a light dosage rate of between 1 mW/cm² and 20 mW/cm².

14. The assembly of claim 12 wherein the light source provides a light dosage rate of between 5 mW/cm² and 20 mW/cm².

15. The assembly of claim 1 wherein the light source provides a light having variable wavelengths that are controlled by a controller.

16. The assembly of claim 1 wherein in the light source is cycled by a controller between an ON state and a substantially OFF state during a treatment protocol utilizing the assembly.

17. The assembly of claim 1 wherein the light source is in its ON state for a period of minutes and then in its substantially OFF period for a period of nanoseconds to hours.

18. The assembly of claim 1 further comprising a surface-acting agent at or near the tissue surface.

19. The assembly of claim 18 wherein the surface-acting agent is provided within the dressing.

20. The assembly of claim 18 wherein the surface-acting agent is provided at or near the tissue surface before or after application of the assembly to the tissue surface.

21. The assembly of claim 18 wherein the surface acting agent contains benzalkonium chloride.

22. The assembly of claim 21 wherein the surface acting agent contains benzalkonium chloride provided in a concentration range of between 0.005% to 0.05%.

23. The assembly of claim 21 wherein the surface acting agent contains polymyxin B sulfate or cetrimide or both.

24. The assembly of claim 23 wherein the energy source includes a battery attached to the assembly.

25. The assembly of claim 24 wherein the battery is controlled by an electronic circuit and/or processor that controls the voltage or current or both applied to the light source so that a light intensity of the light source is generally uniform during application of the assembly at the tissue surface.

26. The assembly of claim 24 wherein the battery is rechargeable through a direct connection or electromagnetic coupling to a remote energy source.

27. The assembly of claim 1 further comprising an electronic circuit or processor for controlling operation of the light source.

28. The assembly of claim 27 wherein the circuit or processor controls the light dose rate or the light intensity or the light wavelengths or a combination thereof.

29. The assembly of claim 1 further comprising an electronic circuit or processor for communicating information associated with the assembly or operation thereof to a remote transceiver.

30. The assembly of claim 1 further comprising a memory element for storing information relating to the assembly or operation thereof.

31. The assembly of claim 27 wherein the electronic circuit or processor is controlled via a remote controller.

32. The assembly of claim 29 wherein the electronic circuit or processor is controlled via a remote controller.

33. The assembly of claim 1 further comprising a fabric element that extends beyond a perimeter of the dressing.

34. The assembly of claim 33 wherein the adhesive element is an adhesive layer between the dressing element and the tissue surface.

35. The assembly of claim 1 further comprising an electrode coupled to a power supply and activated to effect an iontophoretic transfer of a photosensitive material or surfactant into the tissue surface.

36. The assembly of claim 1 wherein the light source provides light having wavelengths of between 380 nm to 900 nm.

37. A method of utilizing the assembly of claim 1 comprising the steps of:
administering a photosensitive material to a tissue site;
adhering the assembly of claim 1 at the tissue site; and
illuminating the tissue site with the light source to provide a therapeutic photodynamic reaction of the photosensitive material at the tissue site.

38. The method of claim 37 wherein the tissue site includes tumor cells or cancer cells or microorganisms or virulence factors or combinations thereof.

39. The method of claim 37 further comprising the step cycling between a period of heightened illumination and a period of substantially reduced illumination.

40. The method of claim 37 further comprising the step of administering a surface-acting agent to the tissue site before or after the step of adhering the assembly at the tissue site.

41. The method of claim 37 further comprising the step of coupling an electrode to a power supply within the assembly to effect an iontophoretic transfer of a photosensitive material or surfactant into the tissue site.

42. A portable assembly adapted to be secured upon a tissue surface comprising:
a source of light powered by a battery;
a flexible dressing adapted to contact a tissue surface, said light passing through at least a portion of the dressing to illuminate the tissue surface, and said light promoting a therapeutic photodynamic reaction of a photosensitive material administered at or near the tissue surface; and
an adhesive element for securing the dressing to the tissue surface.

43. The portable assembly of claim 42 further comprising a light reflector for reflecting light from the light source toward the tissue site.

44. The portable assembly of claim 42 wherein the flexible dressing is a polymer or copolymer or a silicone or a foam or a combination thereof

45. The portable assembly of claim 42 wherein in the light source is cycled between an ON state and a substantially OFF state during a treatment protocol utilizing the portable assembly.

46. The portable assembly of claim 42 further comprising a surface-acting agent at the tissue surface.

47. The portable assembly of claim 42 wherein the surface acting agent is provided within the dressing.

48. The portable assembly of 42 wherein the surface acting agent contains polymyxin B sulfate or cetrimide or benzalkonium chloride or a combination thereof.

49. The portable assembly of claim 42 further comprising an outer fabric element that extends beyond a perimeter of the dressing.

50. The portable assembly of claim 42 further comprising an electrode capable of being coupled to a power supply for effecting an iontophoretic transfer of a photosensitive material or surfactant into the tissue site.

51. The portable assembly of claim 50 wherein the iontophoretic transfer is achieved with a current density of between 0.15-0.60 mA/cm².
